(19) United States
(12) Patent Application Publication
(10) Pub. No.: US 2014/0081348 A1
(43) Pub. Date: Mar. 20, 2014

(54) LOW-FREQUENCY STIMULATION SYSTEMS AND METHODS

(71) Applicant: NeuroPace, Inc., (US)

(72) Inventor: David R. FISCHELL, Fair Haven, NJ (US)

(21) Appl. No.: 13/781,602

(22) Filed: Feb. 28, 2013

Related U.S. Application Data
(60) Provisional application No. 61/618,570, filed on Mar. 30, 2012.

(51) Int. Cl. A61N 1/36; A61N 1/05

(52) U.S. Cl. CPC A61N 1/36064; A61N 1/0529
USPC 607/45

(57) ABSTRACT

Described here are devices and methods for delivering a low-frequency stimulation signal to tissue. In some variations, the low-frequency stimulation signal is delivered using one or more stimulation sequences, in which the low-frequency stimulation is sequentially delivered to a plurality of stimulation pathways. The stimulation pathways may comprise one or more monopolar stimulation pathways and/or one or more bipolar stimulation pathways.

### CROSS-REFERENCED TO RELATED APPLICATIONS

This application claims priority to U.S. Provisional Application Ser. No. 61/618,570, filed on Mar. 30, 2012, which is hereby incorporated by reference in its entirety.

### FIELD

The devices and methods described here are related to systems and methods for providing stimulation to tissue.

### BACKGROUND

Neurostimulation systems, and increasingly implantable neurostimulation systems, may be used to treat various neurological diseases and other neurological disorders, such as epilepsy, movement disorders (e.g., Parkinson's disease), psychological disorders, and chronic pain. A typical neurostimulation system may be configured to generate one or more stimulation signals and may deliver the signals to tissue using one or more electrodes. It may be desirable to provide one or more improvements to stimulation devices and methods.

### BRIEF SUMMARY

Described here are devices and methods for providing neurostimulation. In some variations, a stimulation device may comprise a neurostimulator, a plurality of electrodes, and one or more leads electrically connecting some or all of the electrodes to the neurostimulator. The neurostimulator may comprise a stimulation subsystem, and may be configured to generate one or more stimulation signals. In some variations, the neurostimulator may comprise a first non-responsive stimulation mode. In some of these variations, the first non-responsive stimulation mode may comprise delivering a low-frequency stimulation signal to tissue. The low-frequency stimulation signal may de delivered to tissue via one or more stimulation sequences, in which the low-frequency stimulation signal may be applied sequentially to each of a plurality of stimulation pathways. The plurality of stimulation pathways may comprise one or more monopolar electrodes and/or one or more bipolar electrode pairs. In some variations, a break interval may separate two stimulation sequences. The one or more stimulation sequences, and any intervening break periods, may be repeated continuously, or on a pre-scheduled basis.

The stimulation sequences may comprise sequentially delivering a low-frequency stimulation signal to any number of stimulation pathways. For example, in some variations a stimulation sequence comprises applying the low-frequency stimulation signal to a first stimulation pathway for a first time interval, and applying the low-frequency stimulation signal to a second stimulation for a second time interval following the first time interval. In some of these variations, the stimulation sequence further comprises applying the low-frequency stimulation signal to a third stimulation pathway for a third time interval following the second time interval. In some of these variations, the stimulation sequence further comprises applying the low-frequency stimulation signal to a fourth stimulation pathway for a fourth time interval following the third time interval.

In some variations, the neurostimulators and methods described here may comprise a second non-responsive stimulation mode, in which a high-frequency stimulation signal may be delivered to tissue on a continuous or scheduled basis. In some of these variations, the first and second non-responsive stimulation modes may or may not deliver the low-frequency and high-frequency stimulation signals simultaneously. In some variations the neurostimulators may be configured to detect one or more physiological parameters. In some of these variations, the neurostimulators may comprise a responsive stimulation mode, in which a responsive stimulation signal may be delivered to tissue when the neurostimulator detects one or more predetermined criteria in one or more of the physiological parameters.

### BRIEF DESCRIPTION OF THE DRAWINGS

FIG. 1 shows a block diagram of a variation of a neurostimulator that may provide neurostimulation to a patient

FIG. 2 depicts a perspective view of an implantable neurostimulation system suitable for use with the devices and methods described here.

FIG. 3 shows one variation of a stimulation sequence as applied to a plurality of bipolar stimulation pathways.

FIGS. 4-9 show variations of waveforms suitable for use with the neurostimulation devices and methods described here.

FIG. 10 depicts a flow chart of a method by which a neurostimulator may be configured to deliver a responsive stimulation signal.

### DETAILED DESCRIPTION

Described here are devices and methods for providing neurostimulation. Generally, the neurostimulation may comprise at least a first non-responsive stimulation mode, in which a low-frequency stimulation signal (e.g., a stimulation signal having a primary frequency of 15.0 hertz or less) is delivered to the brain. Generally speaking, a “non-responsive” stimulation signal may be delivered to the brain on a continuous or pre-scheduled basis. In some variations, the neurostimulation may additionally comprise a responsive stimulation mode in which a responsive stimulation signal may be delivered to the brain. A “responsive” stimulation signal may generally be delivered in response to detection of one or more preset criteria (e.g., detection of an impending electrographic event), as will be described in more detail below. Additionally or alternatively, the neurostimulation may comprise a second non-responsive stimulation mode, in which a high-frequency stimulation signal (e.g., a signal having a primary frequency greater than 15 hertz) may be applied to the brain.

As mentioned above, the first non-responsive stimulation mode comprises the delivery of a low-frequency stimulation signal to neural tissue. Generally, the application of a low-frequency stimulation signal may have an inhibitory effect on seizure activity. Long-term stimulation using low-frequency stimulation, however, may result in the formation of potentially-harmful gas bubbles at electrode-tissue interfaces. As such, the first non-responsive stimulation mode comprises delivery of a low-frequency stimulation signal to neural tissue in a manner that may minimize or otherwise prevent the formation of gas bubbles. In some variations, the first non-responsive stimulation mode comprises delivery of a stimulation sequence in which a low-frequency stimulation signal is sequentially applied to each of a plurality stimulation pathways (which may comprise monopolar electrodes or bipolar electrode pairs), such that stimulation signal is applied to one stimulation pathway at a time, as will be described in more detail below.

A stimulation pathway generally comprises one or more electrodes, and may comprise an anode and a cathode such that current may pass through tissue between the anode and the cathode. In some instances, a given electrode may act as an anode or a cathode for multiple stimulation pathways. In other instances, a given electrode may act as an anode or cathode for a single stimulation pathway. Some or all of the electrodes in a stimulation pathway may be electrically connected to a stimulation device via one or more leads. The plurality of stimulation pathways may comprise one or more monopolar stimulation pathways and/or one or more bipolar stimulation pathways. In variations where a stimulation pathway comprises a monopolar stimulation pathway, the stimulation pathway may comprise two electrodes. In these variations, a first electrode may be attached to a stimulation device via one or more leads, and the second electrode may be a reference electrode. The reference electrode may be any suitable electrode, such as, for example, a conductive portion of a housing of the stimulation device. In some variations, a stimulation pathway may be a bipolar stimulation pathway, which may comprise a first electrode and a second electrode which may be electrically connected to a stimulation device via one or more leads. In some of these variations, the first and second electrodes are located on the same lead. In others of these variations, the first and second electrodes are located on different leads. A plurality of stimulation pathways may comprise any combination of monopolar and/or bipolar stimulation pathways. When the stimulation systems deliver a stimulation signal to tissue, the stimulation systems may be configured to select which pathway receives each segment of the stimulation signal, as will be described in more detail below.

Generally, the neurostimulation described here may be delivered to tissue via one or more neurostimulators. The neurostimulator may be configured to generate one or more stimulation signals, and may deliver the stimulation signals to tissue via one or more electrodes. One or more leads may provide an electrical connection between the neurostimulator and the electrodes, which may allow the electrodes to be positioned at different locations in or on the brain. In some variations, the neurostimulator may also be configured to measure one or more physiological parameters from a patient (e.g., electrophysiological signals, temperature, blood pressure, or the like), and may further be configured to detect one or more predetermined criteria (e.g., patterns, neurological conditions) in the measured parameters. In some of these variations, the neurostimulator may be configured to generate one or more responsive stimulation signals upon detection of one or more predetermined criteria. The neurostimulator may be any suitable neurostimulator, such as, for example, one of the neurostimulators described in U.S. Pat. No. 6,690,974 to Archer et al. for “Stimulation Signal Generator for an Implantable Device” and issued Feb. 10, 2004, the contents of which is incorporated herein by reference.

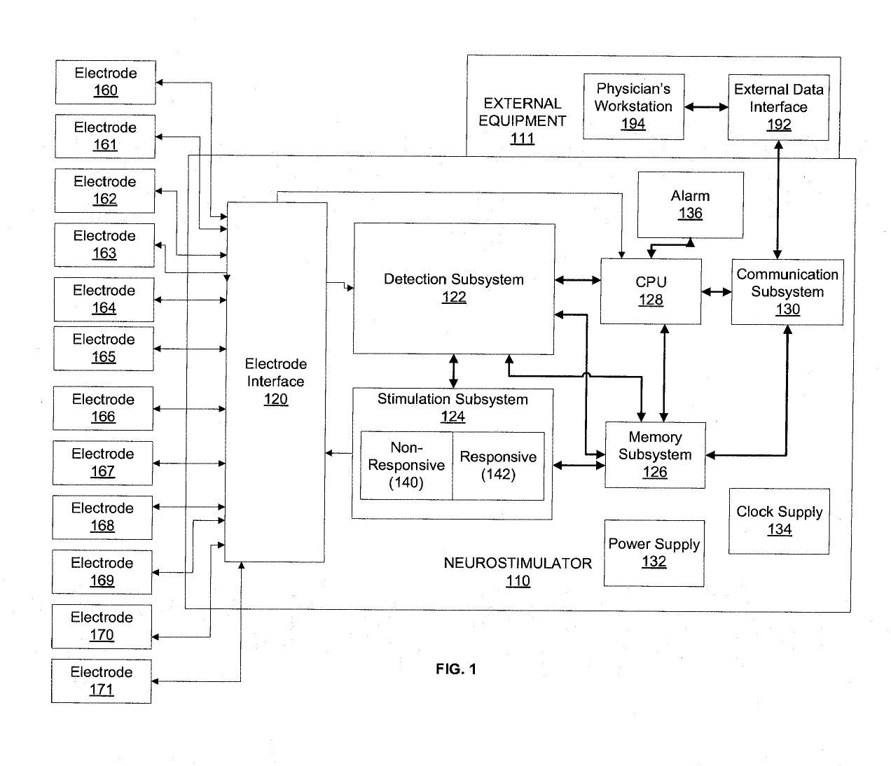

FIG. 1 depicts an overall block diagram of a variation of neurostimulator (110) which may be used to deliver one or more of the stimulation signals mentioned above. As shown there, the neurostimulator (110) may comprise an electrode interface (120), a detection subsystem (122), a stimulation subsystem (124), a memory subsystem (126), a central processing unit (CPU) (128), a communication subsystem (130), a power supply (132), a clock supply (134), and an alarm (136). The neurostimulator (110) may be capable of being coupled to a plurality of electrodes (160)-(171) for sensing and/or stimulation. Each electrode may be configured to sense signals from neural tissue and/or may act as part of a stimulation pathway to deliver stimulation to tissue. It should be appreciated that a particular electrode may be used for sensing and stimulation (e.g., an electrode may be used in a sensing configuration at one point in time, and may be used in a stimulation configuration at a different point in time). Although the neurostimulator (110) is described here with a sensing capability and a subsystem for detecting occurrences of features or events in the sensed signals, neither function is required of a neurostimulator configured to deliver sequential stimulation.

The electrodes (160)-(171) may be connected to neurostimulator (110) via an electrode interface (120). Some or all of the electrodes may be electrically connected to the electrode interface (120) via one or more leads (not shown). The electrode interface (120) may be coupled to the CPU (128), the detection subsystem (122) and the stimulation subsystem (124), and may be configured to select each electrode to act as a sensing electrode, act as an electrode in a stimulation pathway, or remain inactive (e.g., open or shorted). For example, a subset of electrodes of a neurostimulation system may be selected as sensing electrodes, and the electrode interface (120) may at least temporarily connect this subset of electrodes to the detection subsystem (122). This connection may allow detection subsystem (122) to receive one or more electrical signals (e.g., EEG signals, especially electrocorticographic signals (also sometimes referred to as ECoGs) obtained intracranially from the brain) from neural tissue via the sensing electrodes. Additionally or alternatively, a subset of electrodes of a neurostimulation system may be selected where each electrode is a node of a stimulation pathway, and the electrode interface (120) may at least temporarily connect this subset of electrodes to the stimulation subsystem (124), which may allow the stimulation subsystem (124) to selectively deliver stimulation to one or more stimulation pathways. The CPU (128) may control which electrodes are selected as sensing electrodes and stimulation electrodes, and may direct the electrode interface (120) to switch an electrode between sensing, stimulation, or inactive configurations. Additionally, when the neurostimulator (120) is used to deliver a stimulation signal sequentially to a plurality of stimulation pathways, the electrode interface (120) may select which stimulation pathway may receive each portion of the stimulation signal. The electrode interface (120) may also provide one or more additional functions, including, but not limited to, signal amplification, electrode isolation, and charge-balancing functions, but it should be appreciated one or more of these functions may be also be achieved by one or more other subsystems of the neurostimulator. The selection of electrodes to be used in a given stimulation pathway, the segregation of a stimulation signal into different segments for sequential delivery, and the timing of delivery of the different segments may all be determined by programming the neurostimulator with parameters selectable from a menu or range of parameters.

As mentioned above, the neurostimulator (110) may include a detection subsystem (122) which may be configured to measure or otherwise monitor one or more physiological signals or discrete values sensed from a patient. The physiological information may include one or more electrophysiological signals (e.g., EEG signals (especially ECoGs), temperature, blood pressure, and the like. This information may be measured by the neurostimulator via one or more electrodes and/or sensors. For example, in the variation of neurostimulator (110) described above with respect to FIG. 1, the detection subsystem (122) typically comprises an EEG analyzer function which may analyze EEG signals sensed by one or more of the electrodes (160)-(171) (e.g., as selected by the CPU (128) and/or the electrode interface (120)).

In some variations, the detection subsystem (122) may be configured to detect one or more predetermined criteria (e.g., one or more threshold values and/or one or more patterns) in the sensed physiological information. In variations where the neurostimulator comprises a responsive stimulation mode, the neurostimulator may be configured to deliver stimulation (e.g., one or more stimulation signals) when one or more of the predetermined criteria have been detected. The predetermined criteria may comprise one or more patterns, threshold values, or combinations thereof. These criteria may be reflective of an occurring or imminent neurological event. For example, in variations where the detection subsystem (122) comprises an EEG analyzer function, the detection subsystem (122) may receive EEG signals from one or more of the electrodes (160)-(171). The detection subsystem (122) may process and analyze the received EEG signals to identify predefined features or events when these occur (e.g., in the time or frequency domain). The detected feature(s) or event(s) may correspond to or otherwise indicate a seizure, an onset of a seizure, a precursor to a seizure, a symptom of a movement disorder such as a tremor, an episode of depression, a migraine or cluster headache, or the like. EEG signal processing and analysis may comprise one or more signal processing techniques including, but not limited to, half wave counting, line length measurement, and area-under-the-signal calculations. The detection subsystem (122) may comprise one or more of the detection systems described in U.S. Pat. Nos. 6,016,449 to Fischell et al., for “System for Treatment of Neurological Disorders” issued Jan. 18, 2000 and U.S. Pat. No. 6,810,285 to Pless et al. for “Seizure Sensing and Detection Using an Implantable Device” issued Oct. 26, 2004, each of which are hereby incorporated by reference in its entirety.

As mentioned above, the neurostimulator (110) may comprise a stimulation subsystem (124) which may be configured to generate one or more electrical stimulation signals, which may be applied to neural tissue via one or more electrodes. Stimulation subsystem (124) may comprise a non-responsive portion (140) configured to generate one or more non-responsive stimulation signals, either continuously or periodically on a scheduled basis. Additionally or alternatively, the stimulation subsystem (124) may also comprise a responsive portion (142), which may generate one or more responsive stimulation signals when a predetermined criteria has been detected by the detection subsystem (122). It should be appreciated that the stimulation subsystem (124) and the detection subsystem (122) may be in communication (e.g., directly in communication, or in communication via CPU (128)). In some instances, this communication may allow the detection subsystem (122) to blank one or more amplifiers or otherwise filter or process sensed signals during stimulation by the stimulation subsystem (124), as will be described in more detail below. Additionally or alternatively, this communication may allow the stimulation subsystem (124) to alter one or more parameters of a generated stimulation signal based one a signal received by the detection subsystem (122). In addition to the references cited previously, responsive neurostimulation for treating neurological disorders is described in, for example, U.S. Pat. No. 6,459,936 to Fischell et al. for “Methods for Responsively Treating Neurological Disorders” issued Oct. 1, 2002. Multimodal stimulation delivery and devices used to provide it are described in, for example, U.S. Pat. No. 6,466,822 to Pless for “Multimodal Neurostimulator and Process of Using It” issued Oct. 15, 2002 and U.S. Pat. No. 7,174,213 to Pless for “Electrical Stimulation Strategies to Reduce the Incidence of Seizures” issued Feb. 6, 2007. Each of these patents is incorporated by reference in its entirety.

The stimulation subsystem (124) may be programmed to generate any suitable electrical stimulation signals or combination of signals, including pulsatile, non-pulsatile (e.g., sinusoidal or quasi-sinusoidal waveforms), and/or DC signals, as will be described in more detail below. The stimulation subsystem (124) may be configured to provide stimulation signals of any suitable frequency, such as, for example, between about 1 Hz and about 500 Hz. As will be described in more detail below, different modes of neurostimulation may include stimulation signals having different frequencies. The stimulation subsystem (124) may be programmed to generate one or more bursts of pulsatile stimulation. A stimulation output stage of a neurostimulator configurable to generate different varieties of stimulation is described, for example, in U.S. Pat. No. 6,690,974 cited above.

As mentioned above, in variations that include a detection subsystem or function, one or more of the parameters of the stimulation provided by the stimulation subsystem (124) may be specified by one or more other subsystems of the neurostimulator (110). As mentioned above, one or more parameters of the stimulation signal may be determined at least partially by one or more parameters of a signal detected by the detection subsystem (122). U.S. Pat. No. 6,480,743 to Kirkpatrick et al. for “System and Method or Adaptive Brain Stimulation” issued Nov. 12, 2002 and U.S. Pat. No. 6,690,974 cited above, for example, describe methods of using features of a detected signal to determine parameters for stimulation. U.S. Pat. No. 6,480,743 is incorporated by reference in its entirety.

Additionally, some variations of the stimulation subsystem (124) may further be configured to facilitate the administering to a patient one or more additional stimuli, other modulators of neurological activity (e.g., pharmaceuticals), and/or other types of therapy. For example, the stimulation subsystem (124) may be configured to provide a vibratory stimulus, an audio stimulus, and/or may be configured to dispense one or more drugs or therapeutic agents (e.g., via a drug dispenser (not shown)). These additional stimuli may be administered on a non-responsive basis and/or a responsive basis.

As mentioned above, the neurostimulator (110) may also comprise a memory subsystem (126) and a CPU (128), which in some instances may be a microcontroller. In a variation, the memory subsystem (126) may be connected to (i.e., in operable communication with) the detection subsystem (122) and configured to receive or store one or more EEG signals (such as signals sensed before, during, or after a form of stimulation, e.g., a stimulation signal, or an optical signal or electromagnetic or ultrasound therapy) or other data representative of a condition or state of the patient (e.g., a symptom of the disease, the disease itself, or a brain state (sleep or awake states)). The memory subsystem (126) also may be connected to the stimulation subsystem (124) (e.g., for storing and providing programmed stimulation parameters to the stimulation subsystem (124)). The memory subsystem (126) further may be in operable communication with the CPU (128) (e.g., so that the CPU (128) may control the memory subsystem (126)). In variations where the neurostimulator (110) comprises a communication subsystem (130), the memory subsystem (126) may be connected to the communication subsystem (130), which may allow for data stored in the memory subsystem (126) (e.g., data relating to monitored EEG signals, stored EEG signals, stimulation parameters, and the like) to be uploaded to external equipment (111). Additionally, information such as detection criteria and/or stimulation parameters may be downloaded to the memory subsystem (126) from the external equipment (111) via communication subsystem (130).

Similarly, the CPU (128) may be connected to the detection subsystem (122), the stimulation subsystem (124), and/or the communication subsystem (130) for direct control over these subsystems. The CPU (128) may be connected to any suitable subsystem or functional portion of the neurostimulator (110) (e.g., an alarm (136)), and may be configured to control these subsystems and/or functional units. When two or more subsystems or functional units of the neurostimulator (110) are in operable communication, this connection may be analog or digital, and may be achieved using a single wire, a plurality of wires, or wirelessly, or with any other suitable connection mechanism.

As noted above, the neurostimulator (110) may also comprise a communication subsystem (130). The communication subsystem (130) may be coupled to the memory subsystem (126) and/or the CPU (128), and may enable communication between the neurostimulator (110) and the external equipment (111). In the variation shown in FIG. 1, the external equipment (111) may comprise an external data interface (192) and a physician workstation (194). The external data interface (192) and the communication subsystem (130) may be configured to transmit data wirelessly. For example, in some variations the communication subsystem (130) may comprise a telemetry coil (which may or may not be positioned outside of the neurostimulator housing), which may allow for the transmission of signals from the communication subsystem (130) to the external data interface (192), or vice versa, via inductive coupling. Additionally or alternatively, the communication subsystem (130) may comprise an antenna which may provide an RF link between the communication subsystem (130) and the external data interface (192), or may comprise an audio transducer for an audio link.

The external data interface (192) may be coupled to the physician workstation (194) by a communication link, such as a data-transfer cable, a wireless connection, a phone line, an internet connection, or the like. The physician workstation (194) may be configured to upload and receive data from the neurostimulator (110) (e.g., data stored by the memory subsystem (126) corresponding to sensed signals, detected features or events (if the neurostimulator has a detection subsystem or detection functionality), device diagnostics (e.g., remaining power supply voltage, occurrences of device resets, etc.), or data measured in real-time from the sensing elements of the system (e.g., from electrodes configured to sense electrographic activity, such as field potential changes, or from sensors for other physiological information such as temperature, blood pressure, tissue oxygenation, etc.) or features or events detected in real time by the detection subsystem (122)). The physician workstation (194) may have some functionality to undertake various operations on data uploaded from the implanted neurostimulator (110) or other implanted components of a neurostimulation system (e.g., an implanted electrode-bearing deep brain lead or an implanted electrode-bearing cortical strip lead), such as to perform simulations on uploaded data to test whether various detection criteria will result in detecting desired features or events, such as neurological activity corresponding to electrographic onset of an epileptic seizure.

The physician workstation (194) may also be configured to download or transmit from the external equipment (111) to the neurostimulator (110) programming instructions (e.g., stimulation parameter values such as amplitude and frequency of a stimulation signal, number of stimulation pathways to use in a given sequence of stimulation, detection criteria (if the neurostimulator has a detection subsystem or detection functionality), etc.), code and other information to the neurostimulator (110). The physician workstation (194) may further be configurable to command the neurostimulator (110) to perform specific actions (e.g., to record a portion of a monitored electrographic signal) or to change modes (e.g., from a detection-only mode to a responsive stimulation mode, or from a responsive stimulation mode to a scheduled stimulation mode, or from one of these to a combination of the other of these or from one combination to a different combination). Examples of external equipment suitable for use with the neurostimulation devices, systems, and methods describe here may be found in U.S. Pat. No. 6,810,285 to Pless et al. for “Seizure Sensing Device and Detection Using an Implantable Device” issued Oct. 26, 2004 (cited previously herein); U.S. Pat. No. 7,136,695 to Pless et al. for “Patient-Specific Template Development for Neurological Event Detection” issued Nov. 14, 2006; and U.S. Pat. No. 7,277,748 to Wingeier et al. for “Spatiotemporal Pattern Recognition for Neurological Event Detection and Prediction in an Implantable Device” issued Oct. 2, 2007. U.S. Pat. No. 7,819,812 for “Modulation and Analysis of Cerebral Perfusion in Epilepsy and Other Neurological Disorders” issued Oct. 26, 2010, describes external equipment including a programmer (a form of physician workstation) configurable to communicate with a plurality of implanted components (including programmable neurostimulators and leads) and other external equipment (e.g., a database) configurable to communicate with multiple programmers, which external equipment may be beneficially used with the systems, devices and methods described herein. Each of the patents cited above not previously incorporated by reference herein is hereby incorporated by reference in its entirety.

Additionally, the neurostimulator (110) may comprise a power supply (132) for supplying energy to one or more subsystems of the neurostimulator (110). In some variations, the power supply (132) may comprise a primary cell (non-rechargeable) battery. Additionally or alternatively, the power supply (132) may comprise a rechargeable battery. In some variations, the neurostimulator (110) may comprise one or more coils which may receive energy via magnetic induction from an external coil that may be placed in proximity to the coils of the neurostimulator. This energy received from the external coil (which may be positioned outside of the body) may be used to charge a rechargeable battery, or may directly power the neurostimulator (110) during the time the energy is being received by the neurostimulator. In one variation, the external coil may be used to establish a connection with the communication subsystem (130) or the neurostimulator (110). In some variations, one or more of the batteries may be associated with a DC-to-DC converter, which may be used to obtain a voltage larger than the voltage provided by the battery alone (e.g., a compliance voltage for a constant current stimulation output stage of neurostimulator (610)). U.S. Pat. No. 6,690,974 previously cited and incorporated by reference herein, describes such a power supply. As previously described, the neurostimulator (110) may be configured to provide various forms of stimulation, modulation, and therapy. More specifically, with respect to electrical stimulation, the neurostimulator (110) may be configured to supply controlled current (also known as “current-controlled”) stimulation (by keeping a compliance voltage constant), controlled voltage (also known as “voltage-controlled” or “controlled-power”) stimulation, or controlled charge stimulation. See, e.g., Simpson et al., “An Experimental Study of Voltage, Current and Charge Controlled Stimulation Front-End Circuitry,” 2007, IEEE, the contents of which are hereby incorporated by reference herein.

Also shown in FIG. 1 is a clock supply (134). The clock supply (134) may supply any or all of the subsystems and other function units of the neurostimulator (110) with any clock and timing signals that may be necessary for their operation. For example, the clock supply (134) may help synchronize different subsystems within the neurostimulator (110) or may provide time information for time- and date-stamping of neurological data sensed by the system and received, recorded, stored or otherwise obtained by the neurostimulator (110). In some variations, when the detection subsystem (122) of the neurostimulator (110) detects a neurological event or other predefined feature or condition in a sensed signal or other monitored value, the date and time of detection as well as other information relating to the occurrence may be stored in the memory subsystem (126) of the neurostimulator. The CPU (128) and/or the communication subsystem (130) may also provide data to the clock supply (134) to set the correct date and time of the clock supply (134).

While shown in FIG. 1 as having the alarm (136), the neurostimulator (110) need not. In variations that do comprise an alarm (136), the alarm (136) may be any component suitable for providing a perceivable stimulus to the patient (electrical stimulation delivered to neural tissue of the brain is generally not perceptible by a human patient). For example, in some variations the alarm (136) may be configured to deliver a tactile stimulus to the patient (e.g., via vibration), an auditory stimulus (e.g., via a speaker), and/or another somatosensory signal (e.g., an electrical stimulation “tickle”) that may be perceived by the patient. The alarm (136) may be used to notify a patient of one or more conditions. In some variations, the alarm (136) may notify a patient that a particular neurological event has been detected. In other variations, the alarm (136) may notify a patient that the neurostimulator (110) is about to deliver stimulation to the patient. In still other variations, the alarm (136) may notify the patient of a condition of the neurostimulator (110), such as a low or insufficient power supply, a full or nearly full memory subsystem (126), a device failure condition, or the like. It should be appreciated that the alarm (136) may be configured to deliver different stimuli to signify different information to a patient (e.g., a vibratory stimulus may be used to notify a patient of a low or insufficient battery supply, while an acoustic stimulus may be used to notify a patient that a neurological event has been detected). It should also be appreciated that in some variations, a stimulus provided by the alarm (136), such as an acoustic stimulus, may help terminate a neurological event (i.e., the alarm itself may constitute a therapy for a condition, such as aborting an epileptic seizure).

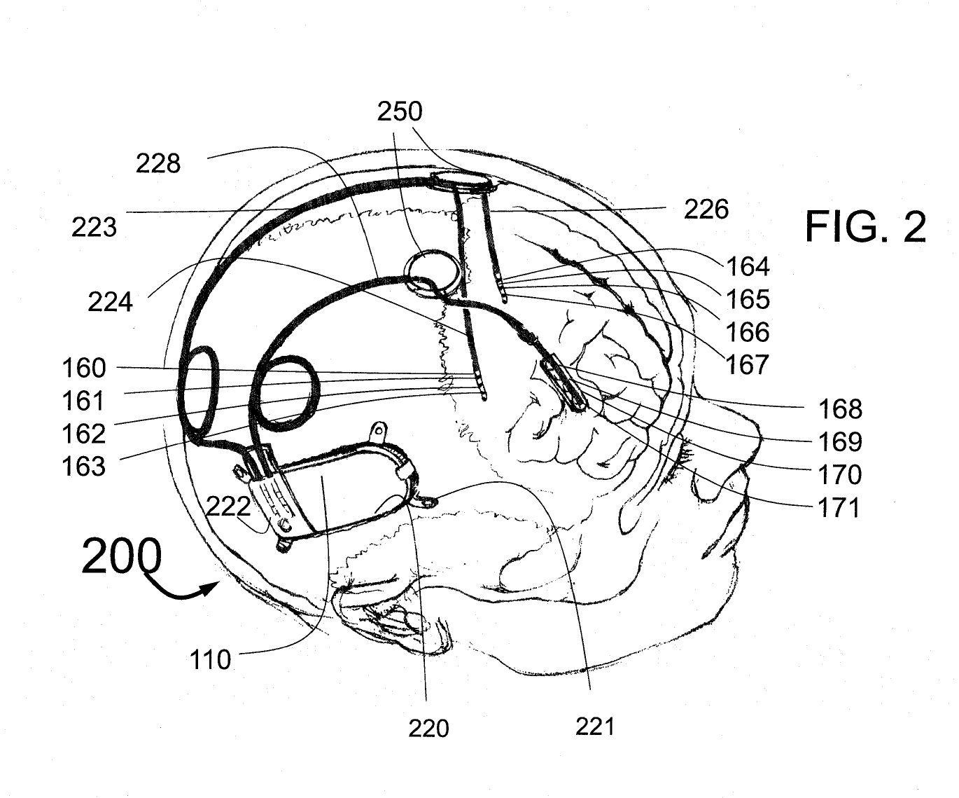

The neurostimulator (110) may be implanted, but need not be. In variations where the neurostimulator (110) is implantable, it may be implanted in any suitable location. In some variations, the neurostimulator (110) may be intracranially implanted. FIG. 2 shows one variation of an implantable neurostimulation system (200) in which the components of the neurostimulator (110) may be contained within an implantable housing (220). The housing (220) may be seated in an opening formed in the patient's cranium (for example, an opening formed by a craniotomy) and may additionally be anchored or otherwise attached to the cranium using one or more fasteners (221) (e.g., one or more bone screws, or the like). Additionally, in the variation shown in FIG. 2, the neurostimulation system (200) may comprise a first bifurcated lead (223) having a first bifurcation (224) and a second bifurcation (226), and a second cortical strip lead (228). The first lead (223) and the second lead (728) may each be coupled to the neurostimulator (110) at a lead connector or a lead interface (222). The two bifurcations (224) and (226) of the first lead (223) and the second lead (228) each may access the brain through one or more burr holes (250) (two burr holes (250) are shown in FIG. 2) formed in the cranium, and may be used to position one or more electrodes (160)-(171) positioned on a distal portion of a lead relative to the brain.

While shown in FIG. 2 as comprising a bifurcated depth lead (223) (with first and second bifurcations (224) and (226)) and a cortical strip lead (228), the neurostimulation system (200) may comprise any suitable number of leads (e.g., one, two, three, or four or more leads). Additionally, each lead may comprise any suitable number of electrodes or other probes or transducers for sensing and/or stimulation or neuromodulation or other therapy. In the variation of neurostimulation system (200) shown in FIG. 2, the first lead (723) may comprise a furcated lead similar to the furcated lead described in U.S. Pat. No. 6,597,953 to Boling et al. for “Furcated Sensing and Stimulation Lead” issued Jul. 22, 2003. A depth lead (also referred to as a “deep brain lead”) typically has one or more electrodes positioned at a distal portion thereof which are designed to contact neural tissue in a region or structure of the brain (e.g., a region comprising or near a hippocampus of the brain). In FIG. 2, the first lead (223) is shown as a bifurcated depth lead with a uniform proximal lead portion and two furcated portions ((224) and (226)). More particularly, the first depth lead (223) of FIG. 2 is shown as provided with four cylindrical electrodes (i.e., the electrodes (160)-(163)) on the first bifurcation (224) and another four cylindrical electrodes (i.e., the electrodes (164)-(167)) on the second bifurcation (226). The portion of lead (223) proximal of the bifurcations (224) and (226) may extend out of one of burr holes (250) and is mechanically and electrically connected to the neurostimulator (110) via the lead connector (222) and conductors (not shown) extending through the lead from the electrodes (160)-(167), respectively. The cortical strip lead (228) may include four disc electrodes (168)-(171) on a proximal portion thereof. A cortical strip lead may be placed so as to position one or more electrodes on a surface of the brain (e.g., the cerebral cortex where it is believed that a focus of a partial epileptic seizure might exist when a patient has a seizure). Although in FIG. 2, a proximal portion of the cortical strip lead (228) is shown as extending out through one of the two burr holes (250), it will be appreciated that cortical strip leads often may be implanted in a patient without using a burr hole (250), and positioned so that one or more electrodes on a distal portion thereof are located under the dura mater and against or adjacent a surface of a patient's brain. Electrode-bearing leads for sensing electrographic activity and providing stimulation (especially electrical stimulation) to a patient's brain are described in U.S. Pat. No. 7,146,222 for “Reinforced Sensing and Stimulation Leads and Use in Detection Systems” issued Dec. 5, 2006. U.S. Pat. No. 6,944,501 to Pless for “Neurostimulator Involving Stimulation Strategies and Process for Using It,” issued Sep. 13, 2005 describes some of the considerations that may be involved in determine where to position electrodes for sensing electrographic information from the brain and delivering electrical stimulation to the brain and the selection of stimulation parameters based on the location of electrodes. Both of these patents are incorporated by reference herein in its entirety. U.S. Pat. No. 7,277,748 to Wingeier et al. for “Spatiotemporal Pattern Recognition for Neurological Event Detection and Prediction in an Implantable Device” issued Oct. 2, 2007 (cited and incorporated by reference previously) describes spatiotemporal considerations when electrodes are used to sense information from the brain. Each lead may be positioned so that the electrodes thereon are situated at any suitable location or locations in the brain. Examples of suitable electrode placement sites may include, but are not limited to the right and left hippocampus, right and left mesial temporal lobe, and the right and left anterior thalamus.

Although twelve electrodes (160)-(171) are shown in FIGS. 1 and 2, it should be recognized that the neurostimulation systems described here may comprise any suitable number of electrodes. For example, in some variations the neurostimulation system may comprise two, three, four, five, six, seven, eight, nine, ten, eleven, twelve, thirteen, fourteen, or fifteen or more electrodes. Additionally or alternatively, one or more portions of the neurostimulator housing (220) may act as an electrode, which may serve as a reference electrode for a monopolar stimulation pathway. For example, the housing (220) may comprise one or more conductive exterior portions, which may act as an electrode in use of the neurostimulator (110). Generally, each electrode may be selected (e.g., via the CPU (128) and/or the electrode interface (120)) and configured to act as either a sensing electrode (e.g., to sense one or more electrical signals the patient) or as an anode or cathode of a stimulation pathway (e.g., to deliver one on or more stimulation signals through an anode/cathode stimulation pathway to the brain). For example, in the variation of the stimulation subsystem (200) shown in FIG. 2 the stimulation system (200) may be programmed and configured to use some or all of the electrodes (160)-(163) of the first bifurcation (224) and the electrodes (164)-(167) of the second bifurcation (226) to from a plurality of stimulation pathways (e.g., one or more monopolar stimulation pathways and/or one or more bipolar stimulation pathways), while the electrodes (168)-(171) of the cortical strip lead (228) may be configured to act as sensing electrodes. The neurostimulation system (200) may be configured to deliver stimulation via a first non-responsive stimulation mode, a second non-responsive stimulation mode, and/or a responsive stimulation mode, each of which will be described in more detail below.

### Non-Responsive Stimulation

As mentioned above, the neurostimulators described here may be configured to deliver one or more non-responsive stimulation signals to the brain. Generally, these stimulation signals are generated and delivered on a continuous or scheduled basis. Parameters of the stimulation signals may be pre-programmed, but may also be at least partially determined by one or more signals or electrophysiological parameters measured from the patient. In some variations, the parameters of the stimulation signal may be varied according to circadian rhythms (e.g., in some patients it may be advantageous to alter stimulation patterns before or during normal sleep times to avoid disrupting sleep patterns).

The neurostimulators and neurostimulation methods described here may comprise a first non-responsive stimulation mode, in which a low-frequency stimulation signal is delivered to a patient. In these variations, the low-frequency stimulation signal may have a primary frequency of 15 Hz or less. In some of these variations, the low-frequency stimulation signal may have a primary frequency of 10 Hz or less. In some of these variations, the low-frequency stimulation signal may have a primary frequency of 2 Hz or less. In some variations the low-frequency stimulation signal may have a primary frequency of 1.1 Hz or less. Generally, the low-frequency stimulation signal may, when delivered to tissue, provide an inhibitory effect on epileptic activity.

Generally, the first non-responsive stimulation mode may be configured to deliver the low-frequency stimulation signal in a manner that may help minimize or otherwise prevent the formation of gas bubbles at a tissue-electrode interface. The first non-responsive stimulation mode may be configured to apply one or more stimulation sequences in which a low-frequency stimulation signal may be temporarily and sequentially applied to each of a plurality of stimulation pathways, such that the stimulation signal is only applied to one stimulation pathway at a time. For example, the stimulation sequence may comprise delivering a low-frequency stimulation signal sequentially to N stimulation pathways. In these variations, the stimulation sequence may comprise apply a low-frequency stimulation signal to a first stimulation pathway for a first time interval, to a second stimulation pathway for a second time interval following the first interval, to a third stimulation pathway for a third time interval following the second interval, and so on, until the stimulation signal is supplied to the Nth stimulation pathway for a Nth time interval following the (N−1)th time interval.

During a stimulation sequence, the low-frequency stimulation signal may be applied to a single stimulation pathway during each time interval. Although the low-frequency stimulation signal may be delivered to a patient for the entire stimulation sequence (which may be providing an inhibitory effect to neural tissue), each stimulation pathway may deliver the low-frequency stimulation signal for a fraction of the overall stimulation sequence. By reducing the time that each stimulation pathway delivers the low-frequency stimulation signal to tissue, the first non-responsive stimulation mode may minimize the formation of gas bubbles at the electrode-tissue interface. Additionally, if gas bubbles or microbubbles do begin to form during the time interval or intervals when the stimulation low-frequency stimulation signal is applied to a particular stimulation pathway, these bubbles or microbubbles may be at least partially resorbed by the body during time intervals when the low-frequency stimulation signal is applied to a different stimulation pathway.

During a stimulation sequence of the first non-responsive stimulation mode, a low-frequency stimulation signal may be sequentially applied to any suitable plurality of stimulation pathways. In some variations, one or more of the plurality stimulation pathways may comprise a monopolar stimulation pathway, in which the stimulation signal passes between an individual electrode (e.g., an electrode on a lead) and one or more reference electrodes (e.g., a conductive portion of a neurostimulator housing). In other variations, one or more of the plurality of stimulation pathways may comprise a bipolar stimulation pathway, in which current passes between an electrode pair on one or more leads. In some variations, each of the electrodes of a bipolar electrode pair may be located on the same lead. In other variations, the electrodes of a bipolar electrode pair may be located on different leads. It should also be appreciated that a stimulation sequence may comprise application of the low-frequency stimulation signal to monopolar and bipolar stimulation pathways. For example, a stimulation sequence may comprise applying a stimulation signal to a monopolar stimulation pathway for a first time interval and applying the stimulation signal to a bipolar stimulation pathway for a second time interval for a second time interval following the first time interval.

The stimulation sequence may be applied to any suitable number of stimulation pathways, such as, for example, two, three, four, five, six, seven, eight, or nine or more stimulation pathways. In some variations, the first non-responsive stimulation mode may be configured to apply a stimulation sequence to two stimulation pathways. In these variations, the stimulation sequence comprises applying a stimulation signal to the first stimulation pathway for a first time interval, and the then applying the stimulation signal to the second stimulation pathway for a second time interval following the first interval. The stimulation sequence may be repeated one or more times, and may be repeated continuously, as will be described in more detail below.

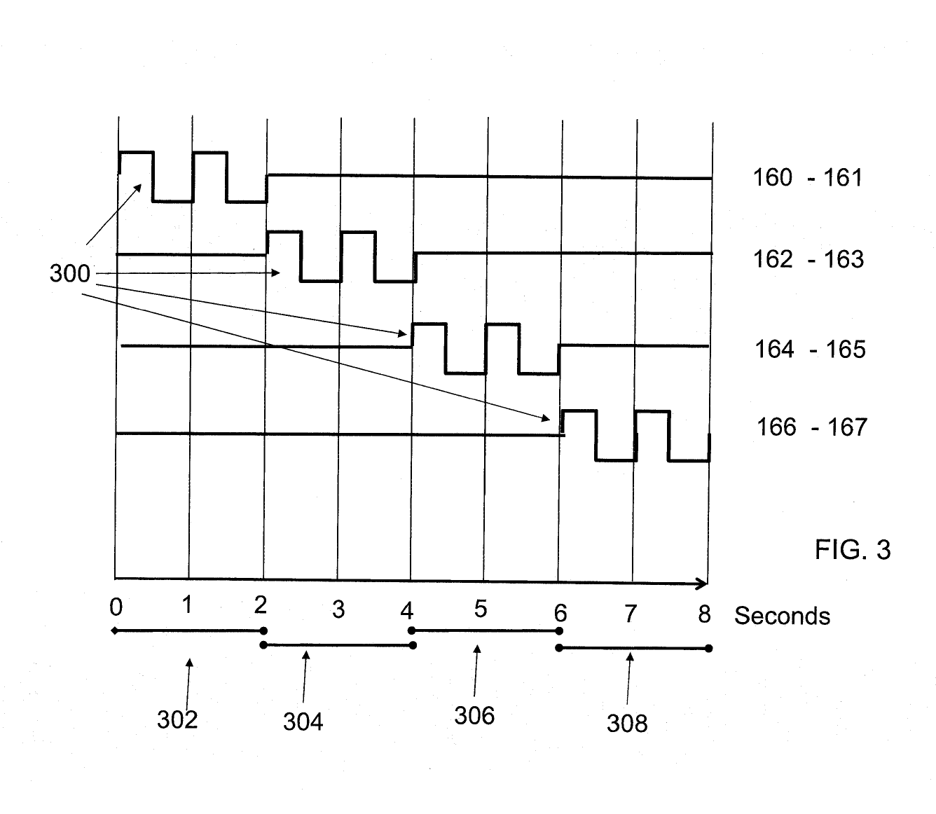

In some variations, the first non-responsive stimulation mode may be configured to provide a stimulation sequence to four stimulation pathways. In these variations, a stimulation sequence may comprise delivering the stimulation signal to a first stimulation pathway for a first time interval, to a second stimulation pathway for a second time interval following the first interval, a third stimulation pathway for a third time interval following the second interval, and a fourth stimulation pathway for a fourth time interval following the third time interval. FIG. 3 shows time graph of one variation of a stimulation sequence that may be applied during a first non-responsive stimulation mode using neurostimulation system (200). As shown there, neurostimulation system (200) may be configured to generate a low-frequency stimulation signal (300) (e.g., via stimulation subsystem (124)), which may be applied to four electrode pairs ((160)-(161), (162)-(163), (164)-(165), and (166)-(167)) during the stimulation sequence. While the stimulation signal (300) shown in FIG. 3 as a 1 Hz square wave, it should be appreciated that low-frequency stimulation signal (300) may be any suitable low-frequency stimulation signal, such as one or more of the signals described in more detail below. During the stimulation sequence, the stimulation signal (300) may be applied to a first electrode pair (160)-(161) on the first bifurcation (224) for a first time interval (302). After the first time interval (302), the stimulation signal (300) may then be applied to a second electrode pair (164)-(165) of the second bifurcation (226) for a second time interval (304). Following the second time interval (304), the stimulation signal (300) may then be applied to a third electrode pair (162)-(163) of the first bifurcation (224) for a third time interval (306). After the third time interval (306), the stimulation signal may then be applied to a fourth electrode pair (166)-(167) of the second bifurcation (226) for a fourth time interval (308). In the variation of stimulation sequence shown in FIG. 3, the stimulation signal (300) may be applied to each electrode pair for 2 seconds, but the stimulation signal (300) is delivered to the patient for 8 seconds. In this way, the first non-responsive stimulation mode may decrease the amount of time each individual electrode pair is providing stimulation (which may reduce the formation of gas bubbles) without decreasing the overall amount of time during which the patient is receiving low-frequency stimulation.

While each of the time intervals (302), (304), (306), and (308) are shown in FIG. 3 as being two seconds, it should be appreciated that the stimulation sequences described here may utilize any suitable time interval. For example, a low-frequency stimulation signal may be applied to an electrode or an electrode pair for a time interval of about 0.5 seconds, about 1 second, about 2 seconds, about 3 seconds, about 4 seconds, or the like. Additionally, while each of the time intervals (302), (304), (306), and (308) are shown in FIG. 3 as being the same value, it should be appreciated that a stimulation sequence may comprise time intervals of different values.

When a stimulation sequence applies a low-frequency stimulation signal sequentially to a plurality of stimulation pathways, the stimulation signal may be applied to the stimulation pathways in any suitable order. In some variations where the stimulation pathways are divided between two or more leads, the stimulation signal may alternate between stimulation pathways on different leads. This may increase the physical distance between the stimulation applied by a stimulation pathway in one time interval and the stimulation applied by a stimulation pathway in the following time interval. For example, in the variation of stimulation sequence shown in FIG. 3, the stimulation signal is applied in an alternating fashion between electrode pairs on the first bifurcation (224) and electrode pairs on the second bifurcation (226) of the lead (223). In other variations, the stimulation sequence may sequentially apply the stimulation signals to the stimulation pathways on a given lead before switching to another lead. For example, in variations where the electrodes are divided between two leads, a stimulation sequence may comprise applying a low-frequency stimulation signal sequentially to the stimulation pathways of the first lead, then applying the low-frequency stimulation signal sequentially to the stimulation pathways of the second lead. For example, using the stimulation pathways identified in FIG. 3, a stimulation sequence may comprise applying a low-frequency stimulation signal to electrode pair (160)-(161) of first bifurcation (224) for a first time interval, then to electrode pair (162)-(163) of the first bifurcation (224) for a second time interval following the first time interval, then to electrode pair (164)-(165) of the second lead for a third time interval following the second time interval, and then to electrode pair (166)-(167) for a fourth time interval following the third time interval. In still other variations, the order of application of the low-frequency stimulation signal may comprise another pre-programmed pattern, or may be randomly generated.

While the stimulation sequence shown in FIG. 3 comprises application of a low-frequency stimulation signal to electrode pairs of the first (224) and second (226) leads, it should be appreciated that a stimulation sequence may comprise application of a low-frequency stimulation signal to any stimulation pathways of any combination of leads in a stimulation subsystem in any suitable order.

The first non-responsive stimulation mode described above may comprise the application of multiple stimulation sequences. For example, in some variations, a first stimulation sequence may be repeated one or more times. In other variations, a subsequent stimulation sequence may be different from the previous stimulation sequence. In some variations, the order of application between the plurality of stimulation pathways may be altered in a subsequent stimulation sequence. In other variations, a subsequent stimulation sequence may apply a low-frequency stimulation signal to a different plurality of stimulation pathways, which may or may not include one or more of the electrodes from the first plurality of stimulation pathways. Additionally or alternatively, one or more time intervals and/or one or more parameters of the stimulation signal may be altered between stimulation sequences.

In variations where a first non-responsive stimulation mode comprises the application of multiple stimulation sequences, the stimulation mode may comprise a break interval between two stimulation sequences. For example, in some variations, a neurostimulator may be programmed to pause stimulation for the duration of the break interval, and then resume stimulation. The break interval may be any suitable period of time, such as, for example, between about 2 seconds and about 10 minutes. In variations in which a break interval separates two stimulation sequences, the break interval may provide an additional time period during which formed gas bubbles or microbubbles may be resorbed by tissue.

In some variations, the first non-responsive stimulation mode may be programmed to insert a break interval between each stimulation sequence. In other variations, break intervals may be inserted after a plurality of stimulation sequences. For example, in some variations a neurostimulator may be configured to supply a stimulation signal via one or more stimulation sequences for a first stimulation interval. Following the first stimulation interval, the first non-responsive stimulation mode may insert a break interval, after which one or more stimulation sequences may be applied for a second stimulation interval. In some variations, the same stimulation sequence may be repeated for the duration of the first and/or second stimulation intervals. For example, in some variations, the 8-second stimulation sequence shown in FIG. 3 may be continuously repeated for a first stimulation interval (e.g., one minute), followed by a break interval, and then may be repeated for one or more subsequent stimulation intervals. In other variations, one or more pre-programmed or randomly generated patterns of stimulation sequences may be applied for the duration of the first and/or second stimulation intervals.

The low-frequency stimulation signal of the first non-responsive stimulation mode may be any suitable signal. The stimulation signal may be pulsatile, non-pulsatile (e.g., sinusoidal or quasi-sinusoidal waveforms), a DC signal, or a combination thereof. In variations where the first signal is pulsatile, the pulses may comprise one or more monophasic or biphasic square pulses. The pulses of the signal may have any suitable pulse width, pulse morphology, pulse-to-pulse interval, pulse amplitude. One or more of these characteristics may be altered between subsequent pulses. The pulses may be voltage controlled or current controlled. The pulses may be biphasic to achieve charge balance, but it should be appreciated that a waveform may comprise a net DC component.

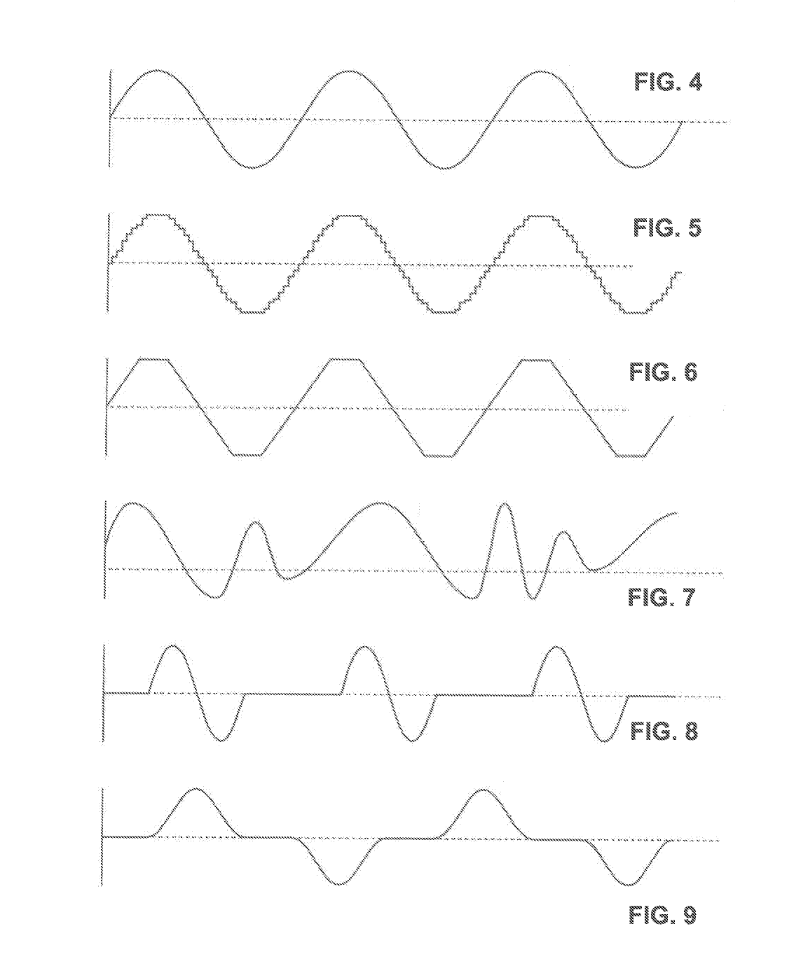

In other variations, the stimulation signal of the first mode may comprise one or more non-pulsatile waveforms. These signals may be any suitable sinusoidal, quasi-sinusoidal waveforms, continuous, semi-continuous, discontinuous, or stepwise approximated waveforms that are not exclusively defined by monophasic or biphasic square pulses. For example, FIG. 4 shows a sinusoidal stimulation signal suitable for use with the first mode. The stimulation may have any suitable amplitude, such as, for example, 0.1 to 10 mA. In some variations, the neurostimulator may be configured to generate a sinusoidal stimulation signal as a stepwise approximation, via a series of small steps as shown in FIG. 5. The time between steps may be any suitable interval (e.g., about 40 microseconds), and may be dependent upon the details of the waveform being generated. It is anticipated that the stair step waveform of FIG. 5 may be filtered to arrive at a waveform more similar to that of FIG. 4, which would allow for longer periods of time between steps and larger steps. Likewise, any of the waveforms described immediately below with respect to FIGS. 6-9 may also be generated as a stepwise approximation, notwithstanding their continuous appearance in the figures.

FIG. 6 depicts a truncated ramp waveform, in which the rate of the ramp, the maximum and minimum amplitudes reached, and the dwell time at the maximums may be selectable parameters. FIG. 7 shows a sinusoidal waveform in which the amplitude and frequency may be varied while the waveform is applied. The amplitude and/or frequency variations may be at least partially pre-programmed, and/or may be at least partially dependent on one or more physiological parameters sensed by the body (e.g., by one or more signals sensed by one or more sensing electrodes during or between stimulation application). While the waveform of FIG. 7 is illustrated as having a positive direct current component, but it should be noted that this waveform, as well as any of the others described herein as suitable for use according to the invention, may or may not be provided with a direct current component as clinically desired.

FIGS. 8-9 depict stimulation signals in which the waveform comprises portions of a continuous waveform that have a selectable delay between these portions. For example, FIG. 7 shows a stimulation signal in which segments of a sine wave separated in time (of course the same technique could be used for the truncated ramp, or other arbitrary morphologies). FIG. 8 shows a stimulation signal comprising haversine pulses, in which the derivative in time of the waveform approaches zero as the amplitude approaches zero. It should be appreciated that these waveform segments may have any suitable morphology, including triangular pulses, trapezoidal pulses, haversine pulses, or combinations thereof.

While the stimulation signals described immediately above may be delivered during a first non-responsive stimulation mode, it should be appreciated that any of these waveforms may be also be utilized in a second non-responsive stimulation mode or a responsive stimulation mode, as will be described in more detail below.

In some variations, the neurostimulation may comprise a second non-responsive stimulation mode, in which a high-frequency stimulation signal is delivered to tissue. In these modes, the stimulation signal may have a primary frequency greater than 15 Hz. In some of these variations, the stimulation signal may have a primary frequency between 15 Hz and 500 Hz. In some of these variations, the stimulation signal may have a primary frequency between 15 Hz and 200 Hz. This high frequency stimulation signal may be delivered to any brain suitable brain structure or structures, such as, for example, the anterior nucleus of the thalamus, or one or more of the location described in more detail above. The high-frequency stimulation signal may comprise one or more of the waveforms (e.g., pulsatile, non-pulsatile, DC) described in more above.

The second non-responsive stimulation mode may be configured to deliver a high-frequency stimulation signal continuously or on a scheduled basis. The second non-responsive stimulation mode may or may not deliver a high-frequency stimulation signal to tissue while the first non-responsive stimulation mode delivers a low-frequency stimulation signal to tissue. In some variations, the high-frequency stimulation signal may be delivered using one or more of the rapid sequential electrode stimulation methods described in U.S. Provisional Patent App. No. 61/618,565, filed on Mar. 30, 2012 and titled “Systems and Methods for Applying Rapid Sequential Electrode Stimulation”, which is hereby incorporated by reference in its entirety.

### Responsive-Stimulation

As mentioned above, in some variations the neurostimulators and neurostimulation methods described here may comprise a responsive stimulation mode in which a stimulation signal is delivered to tissue in response to the detection of one or more criteria by the neurostimulator. For example, responsive stimulation may be initiated when an analysis of the brain's electrical activity shows an impending or existent neurological event, such as epileptiform activity. Generally, this electrical activity may be monitored using one or more of the neurostimulator electrodes and a detection subsystem, such as describe in more detail below. Electrical activity may be detected during the first and/or second non-responsive stimulation mode. Additionally or alternatively, electrical activity detected while stimulation of the non-responsive stimulation modes are paused.

When a neurostimulator detects brain activity during a non-responsive mode of stimulation using one or more sensing electrodes, it may be possible for the sensing electrodes to detect the stimulation signal as it is applied to tissue. This may interfere with the neurostimulator's ability to detect and analyze the brain's electrical activity. Accordingly, it may be desirable to minimize or otherwise prevent detection of the stimulation signal by the neurostimulator. In some cases, the non-responsive stimulation signal is set at a low enough level, and delivered to electrodes that are physically far enough away from the sensing electrodes such that the stimulation does not interfere with detection of brain activity. In variations where the non-responsive stimulation signal comprises a pulsed signal, the neurostimulator may be configured to “blank” the detection amplifier (or other detecting circuit component) during the pulse output of the non-responsive stimulation signal. Generally, the input to some component of the detecting function, such as an amplifier, may be “blanked” for a window that may begin a first period prior to the delivery of the pulse and may end a second period after the pulse ceases. This may prevent the amplifiers from picking up one or more portions of the stimulation signal, so that these stimulation artifacts may not be sensed by the detection subsystem. The neurostimulator may then detect brain activity during the periods in which the detection circuit is being blanked.

In variations where the non-responsive stimulation signal is delivered as a substantially continuous waveform (such as a sinusoidal wave), the use of notch filtering, interference filtering or other continuous time techniques may be used eliminate the non-responsive stimulation artifact from the amplifier used for detecting brain activity. In variations where the neurostimulator employs interference filtering, a portion of the non-responsive stimulation signal may be fed into the inverting input of an error amplifier along with the brain signal. The phase and amplitude of the interfering signal may be adjusted such that it may cancel out the stimulation artifact in the brain signal being sensed. The phase and amplitude may be pre-set in some systems, or a feedback mechanism may be used to drive the energy of the interfering signal towards zero.

When predetermined criteria (e.g., pending or existent epileptiform electrical activity) are detected in some part of the brain during monitoring, a responsive stimulation may be initiated. In some variations, any non-responsive stimulation modes may be paused during delivery of the responsive stimulation signal. In other variations, the responsive stimulation signal may be delivered simultaneously with one or more stimulation signals from a first and/or second non-responsive stimulation mode. In some of these variations, the first and/or second non-responsive stimulation modes may be occasionally paused to determine whether the detected electrical activity has ceased. The responsive stimulation and non-responsive stimulation may be paused simultaneously, or one may cease before the other. If the detected electrical activity has ceased, the responsive stimulation may not be re-initiated, but one or more of the non-responsive stimulation modes may resume. If the detected electrical activity continues, as is discussed below, the responsive stimulation may be re-initiated either with or without being modified in some fashion.

The responsive stimulation may comprise any suitable stimulation signal, such as one or more of the pulsatile, non-pulsatile, or DC signals as described above. In some variations, the responsive stimulation signal may include a burst of pulses. The burst may comprise any suitable number of pulses, such as 1 to 100 or more pulses. After the burst is delivered, the EEG may be re-examined, and if the detected activity was not terminated, a subsequent burst may automatically be delivered. The subsequent burst may have the same signal parameters as the first burst, may re-adapt to the changing EEG rate, or may have new parameters to more aggressively attempt to terminate the epileptiform activity (e.g., higher pulse or burst rate, more pulses, higher amplitude, or modified pulse to pulse intervals, variations on such parameters). Each subsequent burst may be pre-programmed, or may be based on measurements taken from the system. In some variations, the burst or bursts may be delivered using one or more of the rapid sequential electrode stimulation methods described in U.S. Provisional Patent App. No. 61/618,565, filed on Mar. 30, 2012 and titled “Systems and Methods for Applying Rapid Sequential Electrode Stimulation”, which was previously incorporated by reference.

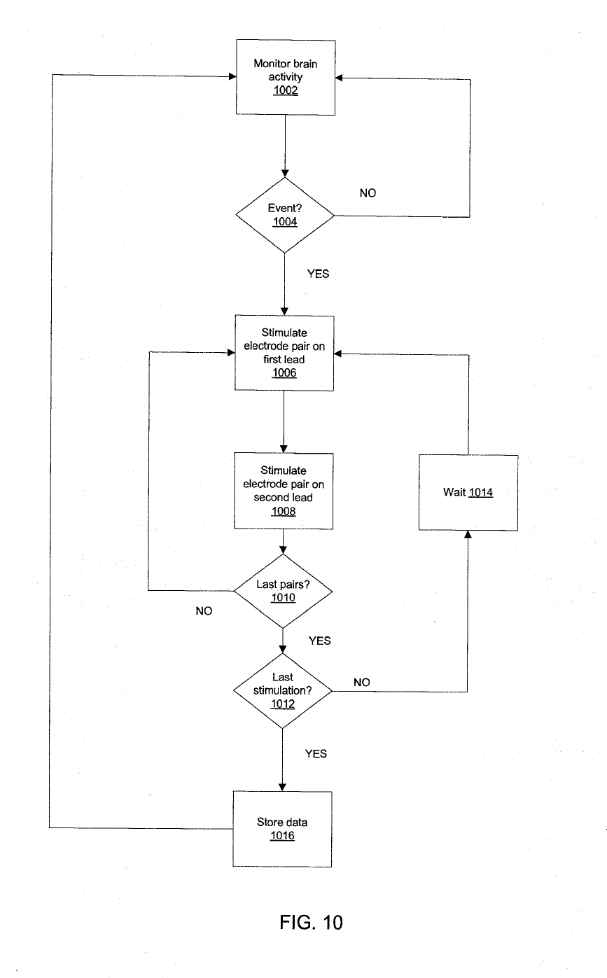

When the responsive stimulation mode provides a responsive stimulation signal, the stimulation signal may be applied to multiple electrodes simultaneously, or may be sequentially applied to a plurality of electrodes in one or more stimulation sequences such as those described in more detail above. For example, FIG. 10 shows a flow chart illustrating a variation of how the neurostimulator (110) described above with respect to FIG. 1 may be configured to detect and treat a neurological event (or other predetermined criteria) using a stimulation sequence. Generally, the neurostimulator (110) may be configured to monitor brain activity (1002). This monitoring may occur continuously, or may occur during pauses in one or more non-responsive stimulation modes. Monitoring brain activity may comprise using detection subsystem (122) to process EEG signals received from one or more of electrodes (160)-(171). These EEG signals may be monitored until the detection subsystem (122) detects an event (1004), such as described in more detail above.

When an event is detected, the detection subsystem (122) may provide a signal to the CPU (128) to indicate that an event has been detected. The CPU (128) may then signal the stimulation subsystem (124) to deliver a stimulation signal via a stimulation subsequence. The stimulation signal may be a high-frequency stimulation signal, a low-frequency stimulation signal, or a combination thereof. In some variations, the stimulation sequence comprises application of the stimulation signal to electrode pairs of the first bifurcation (224) and second bifurcation (226) in an alternating fashion. Specifically, the stimulation signal may be applied to a first electrode pair of the first bifurcation (224) (e.g., electrode pair (160)-(161)) for a first time interval (1006), and then be applied to a first electrode pair of second bifurcation (226) (e.g., electrode pair (164)-(165)) for a second time interval (1008). This may be repeated with subsequent electrode pairs on each of first (224) and second (226) leads, until the stimulation signal has been applied to the last electrode pair on each lead (1010). This stimulation sequence may be repeated one or more times. Additionally, in some variations, the stimulation subsystem (124) may send a signal to the detection subsystem (124) to disable event detection during application of the stimulation signal. Additionally or alternatively, the CPU (128) may also send a signal to the alarm (136) to notify the patient that an event has occurred.

Once the stimulation sequence or sequences have been applied, the detection subsystem (122) may again monitor electrical activity of the brain. If the detection subsystem (122) determines that the detected condition is still present, the stimulation subsystem (124) may deliver one or more additional stimulation sequences to tissue. In some variations, the neurostimulator may wait a period of time before applying a subsequent stimulation sequence (1014). The subsequent stimulation sequences may be the same as or may be different from a preceding stimulation sequence. Subsequent stimulation sequences may be applied to tissue until either a) the detected condition is no longer detected by the detection subsystem (122), or b) a predetermined number of stimulation sequences have been delivered to the patient. Once this last stimulation has been detected (1012), the neurostimulator (110) may be configured to store data (e.g., one or more EEG signals) related to the detected event and the responsive stimulation (1016). This data may be stored in memory subsystem (126), and may later be retrieved via external equipment. Data may be stored for a specific window on either side of the detection of an event (e.g., between about 0.1 minutes and about 30 minutes before and/or after detection of an event). Following stimulation, the neuro stimulator (110) may return to monitoring brain activity.

While particular embodiments and applications of the present invention have been illustrated and described herein, it is to be understood that the invention is not limited to the precise construction and components disclosed herein and that various modifications, changes, and variations may be made in the arrangement, operation, and details of the methods and apparatuses of the present invention without departing from the spirit and scope of the invention as it is defined in the appended claims.

What is claimed as new and desired to be protected by Letters Patent of the United States is:

1. A method for stimulating brain tissue comprising:
generating a low-frequency stimulation signal; and
delivering a first stimulation sequence, wherein the stimulation sequence comprises sequentially applying the low-frequency stimulation signal to a plurality of stimulation pathways in a stimulation sequence, and wherein the low-frequency stimulation signal is only applied to one stimulation pathway at a time.

2. The method of claim 1 wherein the stimulation sequence comprises applying the low-frequency stimulation signal to a first stimulation pathway for a first time interval, and applying the low-frequency stimulation signal to a second stimulation pathway for a second time interval following the first time interval.

3. The method of claim 2 wherein the stimulation sequence further comprises applying the low-frequency stimulation signal to a third stimulation pathway for a third time interval following the second time interval.

4. The method of claim 3 wherein the stimulation sequence further comprises applying the low-frequency stimulation signal to a fourth stimulation pathway for a fourth time interval following the third time interval.

5. The method of claim 1 further comprising delivering a second stimulation sequence.

6. The method of claim 1 further comprising redelivering the first stimulation sequence.

7. The method of claim 6 further comprising waiting a break interval between delivery and re-delivery of the first stimulation sequence.

8. The method of claim 1 wherein the low-frequency stimulation signal has a frequency less than 10 Hz.

9. The method of claim 8 wherein the low-frequency stimulation signal has a frequency less than 2 Hz.

10. The method of claim 1 wherein the plurality of stimulation pathways comprises a monopolar stimulation pathway.

11. The method of claim 1 wherein the plurality of stimulation pathways comprises a bipolar stimulation pathway.

12. A device for stimulation brain tissue comprising:
a neurostimulator;
one or more leads;
a plurality of electrodes defining a plurality of stimulation pathways,
wherein the neurostimulator is configured to generate a low-frequency stimulation signal comprises a first non-responsive stimulation mode in which at least one stimulation sequence is delivered to tissue, wherein the at least one stimulation sequence comprises sequentially applying the low-frequency stimulation signal to a plurality of stimulation pathways in a stimulation sequence, and wherein the low-frequency stimulation signal is only applied to one stimulation pathway at a time.

13. The device of claim 12 wherein the at least one stimulation sequence comprises applying the low-frequency stimulation signal to a first stimulation pathway for a first time interval, and applying the low-frequency stimulation signal to a second stimulation pathway for a second time interval following the first time interval.

14. The device of claim 13 wherein the at least one stimulation sequence comprises applying the low-frequency stimulation signal to a third stimulation pathway for a third time interval following the second time interval.

15. The device of claim 14 wherein the at least one stimulation sequence further comprises applying the low-frequency stimulation signal to a fourth stimulation pathway for a fourth time interval following the third time interval.

16. The device of claim 12 wherein the neuro stimulator is configured to generate a high-frequency stimulation signal, and the neurostimulator comprises a second non-responsive stimulation mode in which the high-frequency stimulation signal is delivered to tissue.

17. The device of claim 12 wherein the neuro stimulator comprises a responsive stimulation mode, in which a responsive stimulation signal is delivered to tissue.
